(19) United States
(12) Patent Application Publication
(10) Pub. No.: US 2016/0030440 A1
(43) Pub. Date: Feb. 4, 2016

(54) ANTI-TUMORAL COMPOSITION COMPRISING A PI3KBETA-SELECTIVE INHIBITOR AND A PI3KALPHA-SELECTIVE INHIBITOR

(71) Applicant: SANOFI, Paris (FR)

(72) Inventors: Héléne BONNEVAUX, Paris (FR); Carlos GARCIA-ECHEVERRIA, Paris (FR); Angela VIRONE-ODDOS, Paris (FR)

(21) Appl. No.: 14/781,890

(22) PCT Filed: Apr. 3, 2014
(86) PCT No.: PCT/EP2014/056696

§ 371 Date: Oct. 1, 2015

(30) Foreign Application Priority Data
Apr. 5, 2013 (EP) 13305448.6

(51) Int. Cl. A61K 31/5377; A61K 31/4439

(52) U.S. Cl. CPC A61K 31/5377; A61K 31/4439

(57) ABSTRACT

The present invention concerns a combination of a PI3Kβ selective inhibitor with a PI3Kα selective inhibitor for use in the treatment of cancer.

The present invention concerns a combination of a PI3Kβ inhibitor with a PI3Kα inhibitor and its pharmaceutical uses thereof.

Phosphoinositide 3-kinases (PI3Ks) are signalling molecules involved in numerous cellular functions such as proliferation, survival and metastasis. PI3Ks are lipid kinases that produce second messenger molecules activating several target proteins including serine/threonine kinases like PDK1 and AKT (also known as PKB). PI3Ks are divided in three classes and the class I comprises four different PI3Ks named PI3K alpha (PI3Kα), PI3K beta (PI3Kβ), PI3K delta and PI3K gamma.

The class I PI3K is divided in two groups: class IA and class IB PI3K. Class IA PI3K is composed of a heterodimer between a p110 catalytic (α, β and δ isoforms) subunit and a p85 regulatory subunit.

PI3Kα and PI3Kβ are ubiquitously expressed and possess the unique feature of being activated by tyrosine kinase receptors. The PI3Kβ are also activated by G protein-coupled receptors (Vanhaesebroeck et al., Annual Review of Biochemistry, vol. 70, 535-602, 2001).

The compound 2-{2-[(2S)-2-methyl-2,3-dihydro-1H-indol-1-yl]-2-oxoethyl}-6-(morpholin-4-yl)pyrimidin-4(3H)-one (here-below compound (I)) is a selective inhibitor of the PI3Kβ isoform. After treatment with this compound, cancer cells with an activated PI3K pathway, as for example PTEN-deficient tumor cells (Phosphatase and TENsin homolog gene, mutated in multiple advanced cancers), typically respond via inhibition of PI3K targets as for example inhibition of the phosphorylation of AKT as well as of AKT downstream effectors, inhibition of tumor cell proliferation and tumor cell death induction.

The compound (2S)-N1-[4-Methyl-5-[2-(2,2,2-trifluoro-1,1-dimethylethyl)-4-pyridinyl]-2-thiazolyl]-1,2-pyrrolidinedicarboxamide (here-below compound (II)) is a selective inhibitor of the PI3Kα isoform, also known as BYL719. After treatment with this compound, cancer cells with an activated PI3K pathway, as for example PIK3CA mutated tumor cells, typically respond via inhibition of PI3K targets as for example inhibition of the phosphorylation of AKT as well as of AKT downstream effectors, inhibition of tumor cell proliferation and tumor cell death induction.

More particularly, it is known that PTEN-deficient tumors are dependent on PI3Kβ signaling but do not depend on the PI3Kα signaling (Susan Wee et al., PNAS, 2008, vol. 105, no 35, p. 13057-13062). However, the PI3Kβ inhibitors are not always sufficient to treat cancer, such as PTEN-deficient cancers.

There is a need to provide alternative and/or improved treatments of cancer, in particular for PTEN-deficient cancers.

In general, there is an ongoing need for more efficacious methods and compositions in the treatment of cancer. There is also a need to provide a treatment of cancer that is more effective in inhibiting tumor cell proliferation and/or enhancing tumor cell death. There is also a need to minimize toxicity towards patients. There is a particular need for PI3Kβ inhibitor therapy used in combination with other targeted therapy leading to more efficiency without substantially increasing, or even maintaining or decreasing, the dosages of the PI3Kβ inhibitor generally used.

It is an object of the present invention to provide a novel combination.

It is an object of the present invention to provide a novel combination for use in the treatment of cancer.

It is an object of the present invention to provide a treatment for cancer which inhibits cancer cell proliferation and survival.

It is a further object of the invention to provide a kit, in particular to treat a patient having cancer.

It is an object of the invention to provide a pharmaceutical composition, in particular to treat a patient having cancer.

It is another object of the invention to provide a method of treatment of cancer.

The present invention thus relates to a combination of a PI3Kβ inhibitor with a PI3Kα inhibitor. In one embodiment, the PI3Kβ is different from the PI3Kα inhibitor.

The invention also relates to the above combination for use in medicine, more particularly for use in the treatment of cancer.

The invention also relates to a kit comprising the above mentioned combination, in particular for its use as mentioned above, for simultaneous, separate or sequential administration.

The invention further relates to a pharmaceutical composition comprising the combination of the invention.

The invention relates to a method of treatment comprising administering the above mentioned combination to a patient having cancer.

In one embodiment according to each object of the invention, PI3Kβ inhibitors are compounds which exhibit an inhibitory effect on the PI3Kβ. More particularly, they generally exhibit an inhibitory effect on PI3Kbeta and moderate or no inhibitory effect on other PI3K isoforms, namely PI3Kalpha, PI3Kdelta and PI3Kgamma.

In one embodiment, they are selective towards PI3Kβ isoform. By “selective PI3Kβ inhibitor” it may be understood the ability of the PI3Kβ inhibitors to affect the particular PI3Kβ isoform, in preference to the other isoforms PI3Kalpha, PI3Kdelta and PI3Kgamma. The PI3Kβ selective inhibitors may have the ability to discriminate between these isoforms, and so affect essentially the PI3Kβ isoform. In one embodiment, the selective PI3Kβ inhibitors are not pan-PI3K inhibitors. In one embodiment, said PI3Kβ inhibitors do not inhibit mTOR.

More particularly, in biochemical and cellular assays, selective PI3Kβ inhibitors may target PI3Kβ isoform with an IC₅₀≦300 nM and may be selective versus other PI3K isoforms, PI3K alpha, PI3K delta and PI3K gamma, with an IC₅₀≧250 nM. In one embodiment, they may exhibit a ratio of inhibition of PI3Kβ versus the others isoforms of at least 2 fold.

In one embodiment, the PI3Kβ inhibitor is chosen among compound (I), AZD8186, and GSK2636771. In one embodiment, the PI3Kβ inhibitor is chosen among compound (I), AZD8186, GSK2636771 and AZD6482. In one embodiment, the PI3Kβ inhibitor is chosen between compound (I) and GSK2636771.

In one embodiment, the PI3Kβ inhibitor has the structural formula (I) as defined below:

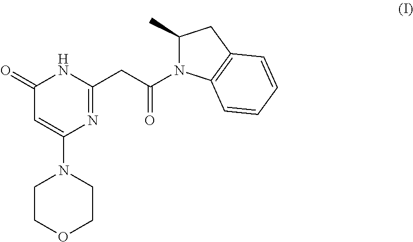 [SMILES: C[C@H]1Cc2ccccc2N1C(=O)Cc1nc(N2CCOCC2)cc(=O)[nH]1]

The PI3Kβ inhibitor according to formula (I) is referred to herein as “compound (I)” The compound (I) is a selective inhibitor of the PI3Kβ isoform of the class I PI3K.

The compound (I) may target PI3Kβ isoform with an IC₅₀ of 65 nM and may be selective versus other PI3K isoforms with an IC₅₀ of 1188 nM, 465 nM and superior to 10 000 nM on PI3Kalpha, PI3Kdelta and PI3Kgamma respectively, in biochemical assays.

The compound (I) may not inhibit mTOR, more particularly may not inhibit mTOR up to 10 μM.

Its selectivity was also controlled by profiling the compound (I) against a large panel of lipid and protein kinases comprising more than 400 kinases. Except PI3Kdelta and PI3Kβ isoform, VPS34 lipid kinase is the only kinase showing an inhibition with a submicromolar IC50 of 180 nM; nevertheless, this level of biochemical activity on VPS34 does not translate in cellular activity using a functional VPS34 cellular assay (IC50 superior to 10,000 nM).

The high level of PI3Kβ-isoform selectivity observed in biochemical settings was confirmed in cellular assays.

In order to specifically explore the compound of formula (I) cellular selectivity against each class I PI3K isoform separately, the inhibition of AKT phosphorylation on serine 473 residue (pAkt-S473) was evaluated in appropriate cellular systems (PIK3CA-mutated H460 lung tumor cells for PI3Kalpha, MEF-3T3-myr p110β mouse fibroblasts overexpressing activated p110β for PI3Kβ, MEF-3T3-myr p110δmouse fibroblasts overexpressing activated p110δ for PI3Kdelta and RAW 264.7 mouse macrophages (after stimulation of AKT phosphorylation by C5a) for PI3Kgamma), as already described (Certal V, Halley F, Virone-Oddos A, Delorme C, Karlsson A, Rak A et al. Discovery and Optimization of New Benzimidazole- and Benzoxazole-Pyrimidone Selective PI3Kβ Inhibitors for the Treatment of Phosphatase and TENsin homologue (PTEN)-Deficient Cancers J. Med. Chem. 2012; 55:4788-4805).

The compound of formula (I) may inhibit PI3Kβ isoform in the PI3Kβ-dependent cell line with a potency 26-fold higher (IC50 of 32 nM) than on PI3Kdelta (IC50 of 823 nM).

The compound of formula (I) may exhibit the same level of activity on PI3Kalpha and PI3Kgamma isoform in cellular and biochemical assays (IC50s of 2,825 and >3,000 nM, respectively).

The compound of formula (I) may be a PI3Kβ-selective inhibitor in cells. The compound of formula (I) may be 26-fold, 88-fold and superior to 94-fold more potent on PI3Kβ than on PI3Kdelta, PI3Kalpha and PI3Kgamma, respectively.

The preparation, properties, and PI3Kβ-inhibiting abilities of compound (I) are provided in, for example, International Patent Publication No. WO2011/001114, particularly Example 117 and Table p 216 therein. The entire contents of WO2011/001114 are incorporated herein by reference. Neutral and salt forms of the compound of formula (I) are all considered herein.

In one embodiment, the PI3Kβ inhibitor is the compound GSK2636771, of formula (III):

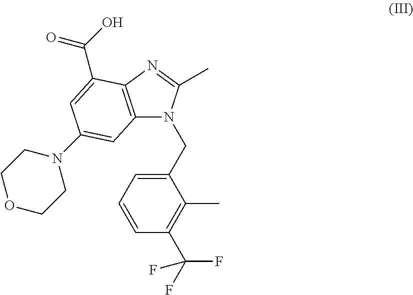 [SMILES: Cc1c(Cn2c(C)nc3c(C(=O)O)cc(N4CCOCC4)cc32)cccc1C(F)(F)F]

The compound GSK2636771, here-below named compound (III), is a selective inhibitor of the PI3Kβ isoform of the class I PI3K as described in “Weigelt B, et al. Clin Cancer Res. 2013, 19(13)” and AACR; Cancer Res 2012; 72(8 Suppl):Abstract nr 1752. The compound (III) may be 12-fold selective over PI3Kdelta and with a >1000 fold selectivity over PI3Kalpha, PI3Kgamma and mTOR. The compound (III) may show less than 30% of inhibition of 294 other kinases at 10 μM. In one embodiment, the compound of formula (III) does not inhibit mTOR.

In one embodiment according to each object of the invention, PI3Kα inhibitors are compounds which exhibit an inhibitory effect on the PI3Kα. More particularly, they generally exhibit an inhibitory effect on PI3Kα and moderate or no inhibitory effect on other PI3K isoforms, namely PI3Kβ, PI3Kdelta and PI3Kgamma.

In one embodiment, they are selective towards PI3Kα isoform. By “selective PI3Kα inhibitor” it may be understood the ability of the PI3Kα inhibitors to affect the particular PI3Kα isoform, in preference to the other isoforms PI3Kbeta, PI3Kdelta and PI3Kgamma. The PI3Kα selective inhibitors may have the ability to discriminate between these isoforms, and so affect essentially the PI3Kα isoform. In one embodiment, the selective PI3Kα inhibitors are not pan-PI3K inhibitors. In one embodiment, said PI3Kα inhibitors do not inhibit mTOR.

More particularly, in biochemical and cellular assays, selective PI3Kα inhibitors may target PI3Kα isoform with an IC₅₀≦250 nM and may be selective versus other PI3K isoforms, PI3Kbeta, PI3Kdelta and PI3Kgamma, with an IC₅₀≧250 nM. In one embodiment, they may exhibit a ratio of inhibition of PI3Kα versus the others isoforms of at least 2 fold.

In one embodiment, the PI3Kα inhibitor is chosen among compound (II), INK-1117 and GDC-0032.

In one embodiment, the PI3Kα inhibitor has the structural formula (II) as defined below:

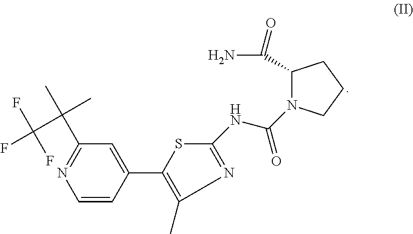 [SMILES: Cc1nc(NC(=O)N2CCC[C@H]2C(N)=O)sc1-c1ccnc(C(C)(C)C(F)(F)F)c1]

The PI3Kα inhibitor according to formula (II) is referred to herein as “compound (II)”. The compound (II) is a selective inhibitor of the PI3Kα isoform of the class I PI3K. The CAS number of compound (II) is 1217486-61-7.

In some embodiments, the compounds described above could be unsolvated or in solvated forms. As known in the art, the solvate can be any of pharmaceutically acceptable solvent, such as water, ethanol, and the like. In general, the presence of a solvate or lack thereof does not have a substantial effect on the efficacy of the PI3Ka or PI3Kβ inhibitor described above.

In some embodiments, these compounds are used in a pharmaceutically acceptable salt form. The salt can be obtained by any of the methods well known in the art, such as any of the methods and salt forms elaborated upon in WO 2011/001114, as incorporated by reference herein.

A “pharmaceutically acceptable salt” of the compound refers to a salt that is pharmaceutically acceptable and that retains pharmacological activity. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, or S. M. Berge, et al., “Pharmaceutical Salts,” J. Pharm. Sci., 1977;66:1-19, both of which are incorporated herein by reference.

Examples of pharmaceutically acceptable acid addition salts include those formed with inorganic acids such as hydrochloric acid, hydrobromic acid, sulfuric acid, nitric acid, phosphoric acid, as well as those salts formed with organic acids, such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-l-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid.

Surprisingly, the inventors discovered that the combination of a PI3Kβ inhibitor together with a PI3Kα inhibitor shows a synergistic effect on the inhibition of the cancer cells proliferation and/or on the induction of the cancer cells death.

More particularly, this synergistic effect is quite surprising on PTEN-deficient tumor cells which are known to be PI3Kα signaling independent (Susan Wee et al., PNAS, 2008, vol. 105, no 35, p. 13057-13062) and for which therefore no effect of PI3Kα inhibitor was expected.

In one embodiment, by synergistic effect, it is understood that the effect of the combination is greater than the expected additive effect of its individual components. More particularly, the synergistic effect may be determined by the ray method design as described in R.Straetemans, (Biometrical Journal, 47, 2005, 299-308).

In another embodiment, by “synergistic effect”, it may also be understood that the effect of the combination is greater than the best effect of one of the two individual components.

In another embodiment, synergy may by defined according to T. H. CORBETT et al., in that a combination manifests therapeutic synergy if it is therapeutically superior to one or other of the constituents used at its optimum dose (T. H. CORBETT et al., Cancer Treatment Reports, 66, 1187 (1982)). According to this definition, to demonstrate the efficacy of a combination, it may be necessary to compare the maximum tolerated dose of the combination with the maximum tolerated dose of each of the separate constituents in the study in question. This efficacy may be quantified, for example by the calculation the log₁₀ cells killed or any other known method.

In one embodiment, the combination of a PI3Kβ inhibitor together with a PI3Kα inhibitor shows a potentiation effect on the inhibition of the cancer cell proliferation. By potentiation effect, it is understood that the effect of the combination is greater than the expected effect of its individual components.

One of the advantages of the present invention is to provide a new treatment for cancer, which may be a targeted therapy in accordance with the expression of some specific genes responsible for an activated PI3K pathway in cancer cells, such as a mutated PTEN gene and/or a mutated PIK3CA gene.

Another advantage of the invention is that thanks to the synergistic effect of the combination as above, lower doses of each active principle may be required to treat cancer and/or drugs toxicity may be reduced.

One of the advantages of the present invention is that the use of isoform specific inhibitors of PI3K allows a reduced toxicity in comparison with the use of pan-PI3K inhibitors (inhibitors which inhibit the four isoforms of PI3K), for which the Dose Limiting Toxicities (DLT) are high and limit their clinical uses.

In one embodiment, synergy according to the invention may be obtained in respect of one of the following effects:
anti-proliferative activity; and/or
cell death induction activity.

In one embodiment, synergy according to the invention may be obtained in respect of one of the following effects:
inhibition of tumor growth (tumor stasis); and/or
partial tumor regression; and/or;
complete tumor regression.

According to an embodiment, the present invention relates to the combination for its use as defined above, wherein the PI3Kβ inhibitor and the PI3Kα inhibitor are in amounts that produce a synergistic effect, as defined above.

In one embodiment, the combination for its use according to the invention enhances anti-proliferative activity and/or pro-apoptotic activity on cancer cells of the patient; more particularly enhances anti-proliferative activity.

In one embodiment, the combination for its use as defined above, can either inhibit tumor cells growth, or achieve partial or complete tumor cells regression.

According to an embodiment, the present invention relates to the combination for its use as defined above, wherein the PI3Kβ inhibitor and the PI3Kα inhibitor are in amounts that produce a synergistic effect and/or a stimulatory effect on the anti-proliferative activity and/or on the pro-apoptotic activity on the cancer cells of the patient. By “stimulatory effect” it may be understood an additive effect according to the ray design method above cited.

In a particular embodiment, said synergistic effect on the anti-proliferative activity may be reached for a ratio PI3Kβ inhibitor/PI3Kα inhibitor comprised from 1/15 to 25/1.

In a particular embodiment, said synergistic effect on the anti-proliferative activity may be reached for a ratio compound (1)/compound (II) comprised from 1/13 to 25/1 in prostate cancer cells and from 1/15 to 24/1 in endometrium cancer cells.

According to an embodiment, the present invention relates to the combination for its use as defined above, wherein the PI3Kβ inhibitor and the PI3Kα inhibitor are in amounts that produce a potentiation effect on the anti-proliferative activity. By “potentiation effect” it may be understood that the effect of the combination is greater than the expected effect of its individual components.

In a particular embodiment, said potentiation effect on the anti-proliferative activity may be reached for the combination at a concentration of PI3Kβ inhibitor of at least 100 nM, with a PI3Kα inhibitor evaluated in a dose-dependent manner.

In a particular embodiment, said potentiation effect on the anti-proliferative activity may be reached for the combination at a concentration of GSK2636771 of at least 100 nM, with the compound (II) evaluated in a dose-dependent manner in prostate cancer cells and in endometrium cancer cells.

Cancers to be treated according to the present invention are chosen from the group consisting of: melanoma, lung cancer, colon cancer, thyroid cancer, prostate cancer, glioblastoma, endometrium and ovarian cancers, breast cancer, gastric cancer and hepatocellularcarcinoma.

More particularly, the cancer is chosen among prostate cancer, glioblastoma, endometrial cancer and breast cancer. More particularly, the cancer is chosen among prostate cancer and endometrial cancer.

For example, the breast cancer may be a triple-negative breast cancer (including BRCA1-associated, basal-like breast tumors). Triple-negative breast cancer is distinguished by negative immunohistochemical assays for expression of the estrogen and progesterone receptors (ER/PR) and human epidermal growth factor receptor-2 (HER2).

In one embodiment, said cancer is characterized by cancer cells with an activated PI3K pathway. In one embodiment, the combination according to the invention is used in the treatment of a patient having cancer cells with an activated PI3K pathway. “Activated PI3K pathway” may refer to cancer cells in which the AKT is phosphorylated as well as AKT downstream effectors, leading to tumor cell proliferation and tumor cell death induction.

In one embodiment, said cancer is characterized by cancer cells which are PTEN-deficient. In one embodiment, the combination according to the invention is used in the treatment of a patient having PTEN-deficient cancer cells. PTEN is a tumor suppressor gene, encoding for the PTEN protein. By “PTEN-deficient cancer cells” it may be understood cancer cells with a PTEN gene exhibiting genetic abnormalities, and/or cancer cells with the partial or complete reduction of PTEN protein expression, for example an inactive PTEN protein, leading to the upregulation of AKT and AKT dowstream effectors by phosphorylation.

In one embodiment, said cancer is characterized by cancer cells which present an activating PIK3CA mutation. In one embodiment, the combination according to the invention is used in the treatment of a patient having cancer cells which present an activating PIK3CA mutation. By “activating PIK3CA mutation”, it may be understood a mutation on the gene PIK3CA which allows the p110α catalytic subunit of PI3K to become constitutively activated.

For example, the activating PIK3CA mutation may be chosen among the E542K mutation, the E545K mutation, the H1047R mutation, the C420R mutation and the R88Q mutation, more particularly the R88Q mutation and the E542K mutation.

In one embodiment, said cancer is characterized by cancer cells which are PTEN-deficient and having an activating PIK3CA mutation. In one embodiment, the combination according to the invention is used in the treatment of a patient having cancer cells which are PTEN-deficient and present an activating PIK3CA mutation.

In one embodiment, the combination according to the invention is used in the treatment of a patient having cancer cells which have a wild type PI3Kα helicoidal domain.

In one embodiment, the combination according to the invention is used in the treatment of a patient having cancer cells which are PTEN-deficient, present an activating PIK3CA mutation and present a wild type PI3Kα helicoidal domain. By “wild type PI3Kα helicoidal domain” is meant a PI3Kα helicoidal domain which does not present a mutation.

In one embodiment, said cancer is characterized by cancer cells which are PTEN-deficient and which are resistant to at least one inhibitor of the tyrosine kinases receptors such as inhibitors of the HER family, the EGFR family etc. . . . In one embodiment, the combination according to the invention is used in the treatment of a patient having cancer cells which are PTEN-deficient and which are resistant to at least one inhibitor of the tyrosine kinases receptors such as inhibitors of the HER family, the EGFR family etc. . . .

By “resistant” it is to be understood that the resistant patient (or resistant cancer cells) to at least one inhibitor of the tyrosine kinases receptors is(are) or was(were) treated by said tyrosine kinases receptors inhibitor and does not respond or respond partially to this treatment (for example, the size of the treated tumor increases) or could respond to the treatment with high and too toxic doses of said tyrosine kinases receptors inhibitor.

In one embodiment according to each object of the invention, a PI3Kβ inhibitor and a PI3Kα inhibitor are in a combined preparation for simultaneous, separate or sequential administration.

According to the invention, “simultaneous” means that the PI3Kβ inhibitor and the PI3Kα inhibitor are administered by the same route and at the same time (eg they can be mixed), “separate” means they are administered by different routes and/or at different times, and “sequential” means they are administered separately, at different times.

Simultaneous administration typically means that both compounds enter the patient at precisely the same time. However, simultaneous administration also includes the possibility that the PI3Kα inhibitor and PI3Kβ inhibitor enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds.

In other embodiments, the PI3Kα and PI3Kβ inhibitors are not simultaneously administered. In this regard, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient.

In a particular embodiment, the administration is separate or sequential and the administration of the PI3Kβ inhibitor is followed by the administration of the PI3Kα inhibitor.

In another particular embodiment, the administration is separate or sequential and the administration of the PI3Ka inhibitor is followed by the administration of the PI3Kβ inhibitor.

In another embodiment, the combined preparation as mentioned above is comprised in a kit, further comprising instructions for use.

In one embodiment according to each object of the invention:
the compound (I), is administered at a dose comprised from 100 to 1600 mg, and
the compound (II), is administered at a dose comprised between 20 and 1600 mg.

More particularly:
the compound (I), is administered at a dose selected from the following doses: 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, and 1600 mg, typically selected from the following doses: 100, 200, 400, 600, 800, 1000, 1200, 1400 and 1600 mg, and
the compound (II), is administered at a dose comprised between 20 and 1600 mg, in particular between 200 and 300 mg.

In one embodiment, the compounds (I) and (II) are administered orally. “Dose” means the administration dose. The dose is not necessarily the “unit dose”, i.e. a single dose which is capable of being administered to a patient, and which can be readily handled and packaged, remaining as a physically and chemically stable unit dose.

In one embodiment, the combination and/or the kit and/or the pharmaceutical composition for their use as mentioned above comprise(s) at least one further anticancer compound.

In one embodiment, the combination and/or the kit and/or the pharmaceutical composition for their use as mentioned above further comprise(s) at least one pharmaceutically acceptable excipient.

In one embodiment, the invention relates to the use of a combination as mentioned above for the preparation of a medicament to treat cancer.

In another aspect, the invention relates to methods of treating a patient with cancer that comprise administering to the patient a therapeutically effective amount of a PI3Kβ inhibitor, in combination with a PI3Kα inhibitor.

In general, the PI3Kβ and PI3Kα inhibiting compounds, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, more particularly in unit dosage forms suitable for simple administration of precise dosages. A particular route of administration is oral, particularly one in which a convenient daily dosage regimen can be adjusted according to the degree of severity of the disease to be treated.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

Dosage forms suitable for parenteral injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms also may comprise buffering agents.

Solid dosage forms as described above can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., PI3Kα or PI3Kβ inhibitor compound described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds described herein with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration may include, for example, ointments, powders, sprays, and inhalants. The active component is admixed under sterile conditions with a physiologically acceptable carrier and any preservatives, buffers, or propellants as can be required. Ophthalmic formulations, eye ointments, powders, and solutions also can be employed.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compounds described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

According to the invention, each of the embodiments can be taken individually or in all possible combinations.

Examples have been set forth below for the purpose of illustration and to describe certain specific embodiments of the invention. However, the scope of the claims is not to be in any way limited by the examples set forth herein.

### BRIEF DESCRIPTION OF THE DRAWINGS

FIG. 1 is an isobologram representation of the in vitro anti-proliferative activity of compound (I) in combination with compound (II) in human prostate cancer cell line PC-3.

FIG. 2 is an isobologram representation of the in vitro anti-proliferative activity of compound (I) in combination with compound (II) in human endometrial cancer cell line HEC-116.

### EXAMPLES

Several in vitro experiments have been conducted in order to study the interaction between a PI3Kβ-selective inhibitor (compound (I)) and a PI3Kα-selective inhibitor (compound (II)) on the inhibitory activity on cell proliferation in human cancer cell lines PC-3 exhibiting a PTEN-deficiency, and HEC-116 exhibiting the two genetic alterations PTEN-deficiency and a PIK3CA mutation (the R88Q mutation).

The interaction between compound (I) and compound (II) on all these cell lines was characterized using ray design approach as described in R. Straetemans, (Biometrical Journal, 47, 2005) which allows to investigate synergy for different effective fraction f of the compounds in the mixture, the effective fraction being constant for each ray. Representative experiments for each combination and each cell line are presented hereunder.

### Example 1

### In Vitro Anti-Proliferative Activity of Compound (I) in Combination with Compound (II) in Human Prostate Adenocarcinoma Cell Line PC-3

To evaluate the anti-proliferative activity of compound (I) in combination with compound (II), experiments were conducted using PTEN-deficient human prostate cancer cell line PC-3. The characterization of the interaction between compound (I) and compound (II) was studied using the ray design method and associated statistical analysis, which evaluates the benefit of the combination at different drug efficacy ratios.

Material and Methods

The human prostate cancer PC-3 cell line was purchased at ATCC (Ref number CRL-1435). The PC-3 cells were cultured in DMEM High Glucose medium supplemented with 10% FBS and 2 mM L-Glutamine.

Compound (I) and compound (II) were dissolved in DMSO at concentration of 30 mM. They were diluted serially, in DMSO following a 3 or 3.3-fold dilution step: then each solution was diluted 50-fold in culture medium containing 10% serum before being added onto cells with a 20-fold dilution factor. The DMSO concentration was 0.1% in controls and in all treated wells.

A ray design method was used allowing the characterization of the interaction of the two compounds for several fixed proportion in the mixture. The ray design includes one ray for each single agent and 19 combination rays. The ray with compound (I) alone has 19 concentrations, the ray with compound (II) alone has 14 concentrations and the combination rays have between 7 and 14 concentrations.

PC-3 cells were plated at 1,000 cells/well in 384-well plates in appropriate culture medium and incubated for 6 hours at 37° C., 5% CO₂. Cells were treated in a grid manner with increasing concentrations of compound (I) ranging from 0.00009 to 30,000 nM and with increasing concentrations of compound (II) ranging from 0.009 to 30,000 nM and incubated for 96 hours. Cell growth was evaluated by measuring intracellular ATP using CelltiterGlo® reagent (Promega) according to the manufacturer's protocol. Briefly, CellTiterGlo® was added to each plate, incubated for 1 hour then luminescent signal was read on the MicroBeta Luminescent plate reader. Three experiments have been performed on the cell line. For each experiment, two 384-well plates were used allowing working with two replicates for combination rays and with four replicates for single agent rays.

Inhibition of cell growth was estimated after treatment with one compound or the combination of compounds for four days and comparing the signal to cells treated with vehicle (DMSO).

Growth inhibition percentage (GI %) was calculated according to the following equation:

GI %=100*(1−((X−BG)/(TC−BG))

where the values are defined as:

X=Value of wells containing cells in the presence of compounds (I) or (II) alone or in combination

BG=Value of wells with medium and without cells

TC=value of wells containing cells in the presence of vehicle (DMSO).

From the growth inhibition percentage, absolute IC40 is defined as the concentration of compound where GI % is equal to 40%.

This measurement allows determining the potential synergistic combinations using the statistical method described hereunder.

The relative potency ρ is first estimated as

$$ρ = \frac{IC {40}_{( 1 )}}{IC {40}_{( 2 )}}$$

where IC40(₁) is the IC40 of the compound (I) and IC40(₂) is the IC40 of the compound (II).

This effective fraction for the ray i is then calculated as

$${f}_{i} = \frac{1}{{c}_{i} \cdot ρ + 1} where {c}_{i} = \frac{[ ( 2 ) ]}{[ ( 1 ) ]}$$

is the constant ratio of the concentrations of the compounds (I) and (II) in the mixture.

A global non linear model using NLMIXED procedure of the software SAS V9.2 was applied to fit simultaneously the concentration-responses curves for each ray. The model used is a 4-parameter logistic model corresponding to the following equation:

$${Y}_{ikj} = E {min}_{i} + \frac{( E {max}_{i} - E {min}_{i} )}{1 + exp [ - {m}_{i} log ( \frac{{Conc}_{ij}}{IC {50}_{i}} ) ]} + {ɛ}_{ijk}$$

Yᵢⱼₖ is the percentage of inhibition for the k^{th} replicate of the j^{th} concentration in the i^{th} ray Concᵢⱼ is the j^{th} mixture concentration (sum of the concentrations of compound (I) and compound (II)) in the i^{th} ray

Eminᵢ is the minimum effect obtained from i^{th} ray

Emaxᵢ is the maximum effect obtained from i^{th} ray

IC50ᵢ is the IC50 obtained from i^{th} ray

mᵢ is the slope of the curve adjusted with data from i^{th} ray

εᵢⱼₖ is the residual for the k^{th} replicate of the j^{th} concentration in the i^{th} ray, εⱼₖ˜N(0, σ²)

Emin, Emax and/or slope were shared whenever it was possible without degrading the quality of the fit.

The combination index Ki of each ray and its 95% confidence interval was then estimated using the following equation based on the Loewe additivity model:

$$\frac{{C}_{( 1 )}}{IC {40}_{( 1 )}} + \frac{{C}_{( 2 )}}{IC {40}_{( 2 )}} = {K}_{i}$$

where IC40₍₁₎ and IC40₍₂₎ are the concentrations of compound (I) and compound (II) necessary to obtain 40% of inhibition for each compound alone and C₍₁₎ and C₍₂₎ are the concentrations of compound (I) and compound (II) in the mixture necessary to obtain 40% of inhibition.

Additivity was then concluded when the confidence interval of the combination index (Ki) includes 1, significant synergy was concluded when the upper bound of the confidence interval of Ki is less than 1 and significant antagonism was concluded when the lower bound of the confidence interval of Ki is higher than 1.

The isobologram representation permits to visualize the position of each ray according to the additivity situation represented by the line joining the point (0,1) to the point (1,0). All rays below this line correspond to a potential synergistic situation whereas all rays above the line correspond to a potential antagonistic situation.

Results of In Vitro Studies

Compound (I), as single agent, inhibited the proliferation of PC-3 cells with an IC40 of 20,200 nM. Compound (II), as single agent, inhibited the proliferation of PC-3 cells with an IC40 of 14,700 nM (see table 1 below).

TABLE 1
Absolute IC₄₀ estimations for each compound alone in example 1
Absolute IC₄₀ of single agents are estimated with a 4-parameter logistic
model

| | Absolute IC40s (nM) |
|---|---|
| Compound (I) | 20,200 [10,700; 38,000] |
| Compound (II) | 14,700 [10,700; 20,200] |

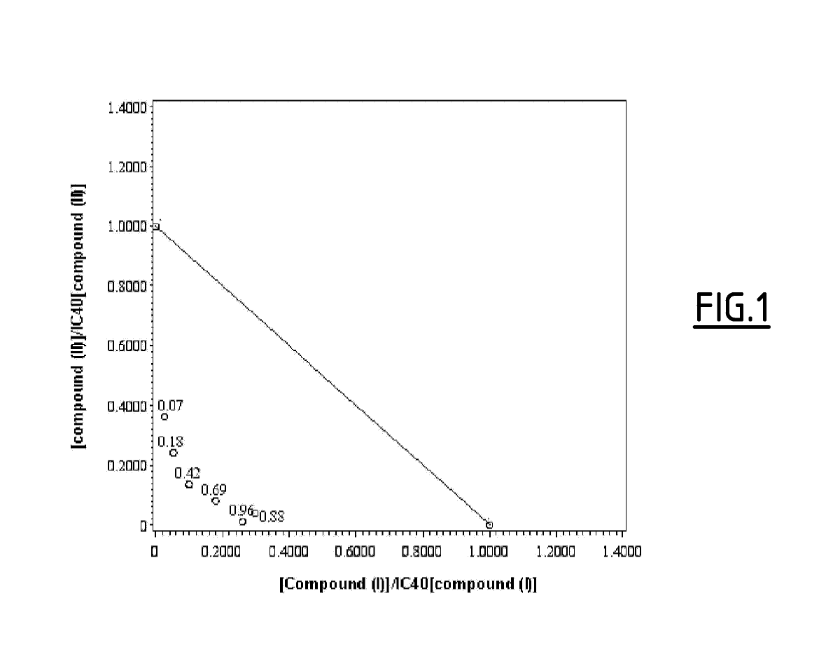

From the isobologram representation (FIG. 1) and the Table 2, significant synergy is observed with a Ki ranging from 0.24 to 0.39 for effective fraction f of compound (I) in the mixture between 0.07 and 0.96 which correspond to the situation where compound (I) is equally, less or more present than compound (II) in the mixture.

TABLE 2
Interaction characterization in example 1
Interaction indexes (Ki) allow us to define the interaction observed
between the two compounds.

| | | Ki (confidence | Interaction |
|---|---|---|---|
| | f values | interval at 95%) | characterization |
| Ray 5 | 0.96 | 0.273 [0.1105; 0.6746] | Synergy |
| Ray 6 | 0.88 | 0.3387 [0.1469; 0.7806] | Synergy |
| Ray 7 | 0.69 | 0.2619 [0.138; 0.4972] | Synergy |
| Ray 8 | 0.42 | 0.237 [0.1409; 0.3989] | Synergy |
| Ray 9 | 0.18 | 0.2964 [0.189; 0.4649] | Synergy |
| Ray 10 | 0.07 | 0.3902 [0.2454; 0.6202] | Synergy |

These data correspond to a representative study out of 3 independent experiments. For these three experiments, synergy was observed for an effective fraction f between 0.05 and 0.98.

### Example 2

### In Vitro Anti-Proliferative Activity of Compound (I) in Combination with Compound (II) in Human Endometrial Carcinoma Cell Line HEC-116

To evaluate the anti-proliferative activity of compound (I) in combination with compound (II), experiments were conducted using human endometrium adenocarcinoma cell line HEC-116 (PTEN-deficient and PIK3CA mutated). The characterization of the interaction between compound (I) and compound (II) was studied using the ray design method and associated statistical analysis, which evaluates the benefit of the combination at different drug efficacy ratios.

### Material and Methods

The human endometrium adenocarcinoma cell line HEC-116 cell line was purchased at JCRB (Ref number JCRB1124 Batch 11072005). The HEC-116 cells were cultured in MEMa medium supplemented with 15% FBS and 2 mM L-Glutamine.

Compounds (I) and (II) dilutions were prepared according to the material and methods of example 1. The final concentrations tested were defined by ray design method described below. The DMSO concentration was 0.1% in controls and in all treated wells.

A ray design method was used allowing the characterization of the interaction of the two compounds for several fixed proportion in the mixture. The ray design includes one ray for each single agent and 19 combination rays. The ray with compound (I) alone has 18 concentrations, the ray with compound (II) alone has 14 concentrations and the combination rays have between 7 and 14 concentrations.

HEC-116 cells were plated at 3,000 cells/well in 384-well plates in appropriate culture medium and incubated for 6 hours at 37° C., 5% CO₂. Cells were treated in a grid manner with increasing concentrations of compound (I) ranging from 0.00009 to 30,000 nM and with increasing concentrations of compound (II) ranging from 0.009 to 30,000 nM and incubated for 96 hours. Cell growth was evaluated by measuring intracellular ATP using CelltiterGlo® reagent (Promega) according to the manufacturer's protocol. Briefly, CellTiterGlo® was added to each plate, incubated for 1 hour then luminescent signal was read on the MicroBeta Luminescent plate reader.

Three experiments have been performed on this cell line. For each experiment, two 384-well plates were used allowing working with two replicates for combination rays and with four replicates for single agent rays.

Inhibition of cell growth was estimated after treatment with single compounds or combination of compounds for four days and comparing the signal to cells treated with vehicle (DMSO) and following equation described in example 1.

These measurements allow determining the potential synergistic combinations in using the statistical method described in example 1.

Results of In Vitro Studies

Compound (I), as single agent, inhibited the proliferation of HEC-116 cells with an IC40 of 12,400 nM. Compound (II), as single agent, inhibited the proliferation of HEC-116 cells with an IC40 of 8,630 nM (see table 3 below).

TABLE 3
Absolute IC₄₀ estimations for each compound alone in example 2
Absolute IC₄₀ of single agents are estimated with a 4-parameter
logistic model

| | Absolute IC₄₀ (nM) |
|---|---|
| Compound (I) | 12,400 [10,400; 14,700] |
| Compound (II) | 8,630 [6,850; 10,900] |

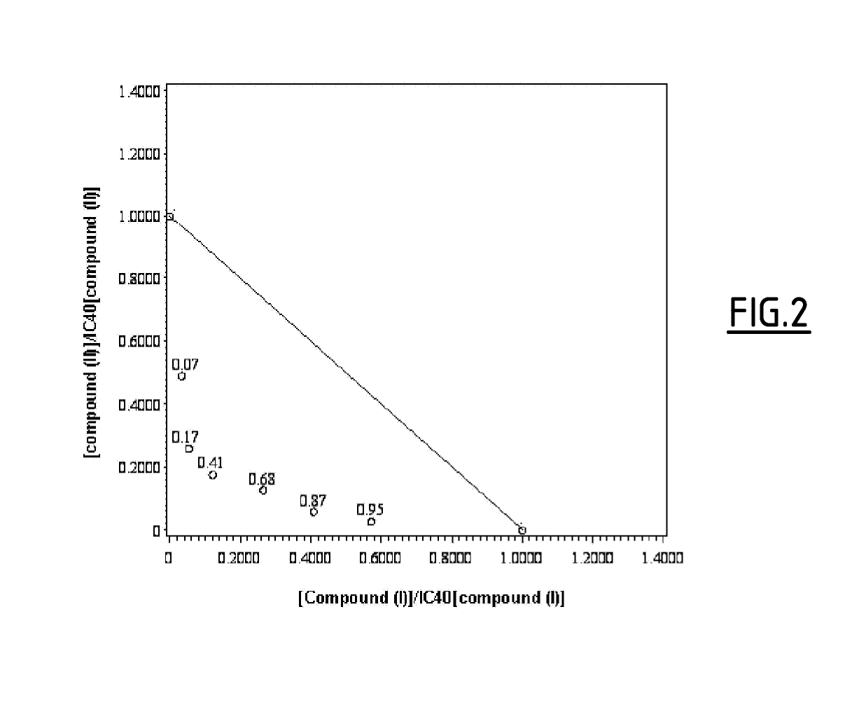

From the isobologram representation (FIG. 2) and the Table 4, significant synergy is observed with a Ki ranging from 0.30 to 0.60 for effective fraction f of compound (I) in the mixture between 0.07 and 0.95.

TABLE 4
Interaction characterization in example 2
Interaction indexes (Ki) allow us to define the interaction
observed between the two compounds.

| | | | Interaction |
|---|---|---|---|
| | f values | Ki (confidence interval at 95%) | characterization |
| Ray 5 | 0.95 | 0.5996 [0.4228; 0.8504] | Synergy |
| Ray 6 | 0.87 | 0.4676 [0.3372; 0.6483] | Synergy |
| Ray 7 | 0.68 | 0.3937 [0.2875; 0.5393] | Synergy |
| Ray 8 | 0.41 | 0.2989 [0.205; 0.4359] | Synergy |
| Ray 9 | 0.17 | 0.3134 [0.226; 0.4346] | Synergy |
| Ray 10 | 0.07 | 0.5251 [0.365; 0.7555] | Synergy |

These data correspond to a representative study out of 3 independent experiments.

For these 3 experiments, synergy was observed for all the effective fractions f of compound (I) in the mixture between 0.03 and 0.96.

Summary of In Vitro Results (Examples 1 and 2)

Interestingly, by the above data, it is demonstrated that a selective PI3Kβ inhibitor (compound (I)) can synergize with a PI3Kα selective inhibitor (compound (II)) to increase the inhibitory activity on cell proliferation in cancer cells exhibiting PI3K pathway activation through PTEN deficiency with or without the co-occurrence of PIK3CA mutation.

FIGS. 1 and 2: Isobologram Representation of Example 1 and 2: In Vitro Anti-Proliferative Activity of Compound (I) in Combination with Compound (II) in Human Cancer Cell Lines PC-3 and HEC-116.

The isobologram representation permits to visualize the position of each ray according to the additivity situation represented by the line joining the point (0,1) to the point (1,0). All rays below this line correspond to a potential synergistic situation whereas all rays above the line correspond to a potential antagonistic situation.

For example 1 experiment, according to the isobologram representation, rays with an effective fraction f between 0.07 and 0.96 are below the additivity line demonstrating significant synergy (see FIG. 1).

For example 2 experiment, according to the isobologram representation, rays with an effective fraction f between 0.07 to 0.95 are below the additivity line with demonstrating significant synergy (see FIG. 2).

1. A combination of a PI3Kbeta inhibitor with a PI3Kalpha inhibitor.

2. The combination according to claim 1, wherein the PI3Kbeta inhibitor is of formula I: 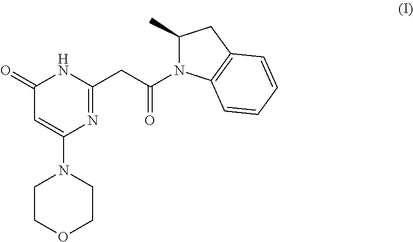 [SMILES: C[C@H]1Cc2ccccc2N1C(=O)Cc1nc(N2CCOCC2)cc(=O)[nH]1]
or one of its pharmaceutically acceptable salts.

3. The combination according to claim 2, wherein the PI3Kalpha inhibitor is of formula II: 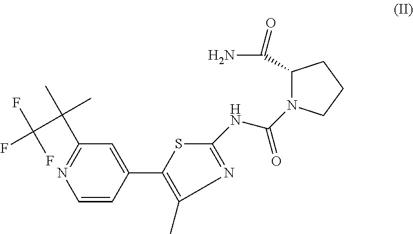 [SMILES: Cc1nc(NC(=O)N2CCC[C@H]2C(N)=O)sc1-c1ccnc(C(C)(C)C(F)(F)F)c1]
or one of its pharmaceutically acceptable salts.

4. A method for treating a disease comprising administering to a patient in the need thereof the combination according to claim 1.

5. A method for treating cancer comprising administering the combination according to claim 1, to a patient with cancer in the need thereof.

6. The method according to claim 5, wherein the cancer is selected from the group consisting of melanoma, lung cancer, colon cancer, thyroid cancer, prostate cancer, glioblastoma, endometrium cancer, ovarian cancer, breast cancer, gastric cancer and hepatocellularcarcinoma.

7. The method according to claim 5, wherein the cancer is selected from the group consisting of prostate cancer, glioblastoma, endometrial cancer and breast cancer.

8. The method according to claim 5, wherein the cancer is characterized by cancer cells which are PTEN deficient.

9. The method according to claim 5, wherein the cancer is characterized by cancer cells which present an activating PIK3CA mutation.

10. The method according to claim 5, wherein the administration of the PI3Kbeta inhibitor and the PI3Kalpha inhibitor is a simultaneous, a separate or a sequential administration.

11. The method according to claim 10, wherein the administration is separate or sequential and wherein the administration of the PI3Kbeta inhibitor is followed by the administration of the PI3Kalpha inhibitor.

12. The method according to claim 10, wherein the administration is separate or sequential and wherein the administration of the PI3Kalpha inhibitor is followed by the administration of the PI3Kbeta inhibitor.

13. A pharmaceutical composition comprising the combination according to claim 1, and at least one pharmaceutically acceptable excipient.

14. A kit comprising the combination according to claim 1.
